SEQUENCE LISTING (23 sequences)

SEQ ID NO: 1 - DNA, 2049 bp, Artificial Sequence
  HL490 STF2R3.HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaatcctcctctacgggcgaaatctgcgacagcccgcatcagattctggatggcaaaaactgcaccctgattgatgcgctgctgggcgatccgcagtgcgatggctttcagaacaaaaaatgggatctgtatgtggaacgcagcagcgcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatgtttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaagatacactggaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 2 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQSSSTGEICDSPHQILDGKNCTLIDALLGDPQCDGFQNKKWDLYVERSSAYSNCYPYDVPDYASLRSLVASSGTLEFNNESFNWTGVTQNGTSSACIRRSKNSFFSRLNWLTHLNFKYPALNVTMPNNEQFDKLYVWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQDTLEAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 3 - DNA, 2157 bp, Artificial Sequence
  HL565 STF2R3.HA1s
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctcacgctgtgcccaacggtaccctggtgaagaccatcaccgacgaccagatcgaggtgaccaacgctaccgagctggtgcagtcctcctccaccggtaagatctgcaacaacccccaccgtatcctggacggtatcgactgcaccctgatcgacgctctgctgggtgacccccactgcgacgtgttccagaacgagacctgggacctgtacgtggagcgttccagcgctttctccaactgctacccctacgacgtgcccgactacgcttccctgcgttccctggtggcttcctccggtaccctggagttcatcaccgagggtttcacctggaccggtgtgacccagaacggtggttccaacgcttgcaagcgtggtcccggttccggtttcttctcccgtctgaactggctgaccaagtccggttccacctaccccgtgctgaacgtgaccatgcccaacaacgacaacttcgacaagctgtacgtgtggggtgtccaccacccctccaccaaccaggagcagacctccctgtacgtgcaggcttccggtcgtgtgaccgtgtccacccgtcgttcccagcagaccatcatccccaacatcggttcccgtccctgggtgcgtggtctgtcctcccgtatctccatctactggaccatcgtgaagcccggtgacgtgctggtgatcaactccaacggtaacctgatcgctccccgtggttacttcaagatgcgtaccggtaagtcctccatcatgcgttccgacgctcccatcgacacctgcatctccgagtgcatcacccccaacggttccatccccaacgacaagcccttccagaacgtgaacaagatcacctacggtgcttgccccaagtacgtgaagcagaacaccctgaagctggctaccggtatgcgtaacgtgccggaaaaggagaccgcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 4 - protein, 714 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAHAVPNGTLVKTITDDQIEVTNATELVQSSSTGKICNNPHRILDGIDCTLIDALLGDPHCDVFQNETWDLYVERSSAFSNCYPYDVPDYASLRSLVASSGTLEFITEGFTWTGVTQNGGSNACKRGPGSGFFSRLNWLTKSGSTYPVLNVTMPNNDNFDKLYVWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKETAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 5 - protein, 709 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAHAVPNGTLVKTITDDQIEVTNATELVQSSSTGKICNNPHRILDGIDCTLIDALLGDPHCDVFQNETWDLVERSAFSNCYPYDVPDYASLRSLVASSGTLEFITEGFTWTGVTQNGGSNACKRGPGSGFFSRLNWLTKSGSTYPVLNVTMPNNDNFDKLYWGHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKTAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 6 - DNA, 2049 bp, Artificial Sequence
  HL566 STF2R3.HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtgcagtcctcctccaccggtaagatctgcaacaacccccaccgtatcctggacggtatcgactgcaccctgatcgacgctctgctgggtgacccccactgcgacgtgttccagaacgagacctgggacctgtacgtggagcgttccagcgctttctccaactgctacccctacgacgtgcccgactacgcttccctgcgttccctggtggcttcctccggtaccctggagttcatcaccgagggtttcacctggaccggtgtgacccagaacggtggttccaacgcttgcaagcgtggtcccggttccggtttcttctcccgtctgaactggctgaccaagtccggttccacctaccccgtgctgaacgtgaccatgcccaacaacgacaacttcgacaagctgtacgtgtggggtgtccaccacccctccaccaaccaggagcagacctccctgtacgtgcaggcttccggtcgtgtgaccgtgtccacccgtcgttcccagcagaccatcatccccaacatcggttcccgtccctgggtgcgtggtctgtcctcccgtatctccatctactggaccatcgtgaagcccggtgacgtgctggtgatcaactccaacggtaacctgatcgctccccgtggttacttcaagatgcgtaccggtaagtcctccatcatgcgttccgacgctcccatcgacacctgcatctccgagtgcatcacccccaacggttccatccccaacgacaagcccttccagaacgtgaacaagatcacctacggtgcttgccccaagtacgtgaagcaggacaccctggaggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 7 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQSSSTGKICNNPHRILDGIDCTLIDALLGDPHCDVFQNETWDLYVERSSAFSNCYPYDVPDYASLRSLVASSGTLEFITEGFTWTGVTQNGGSNACKRGPGSGFFSRLNWLTKSGSTYPVLNVTMPNNDNFDKLYVWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQDTLEAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 8 - DNA, 2049 bp, Artificial Sequence
  HL615 STF2R3 HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaatcctcctctacgggcggtatctgtgatagcccacaccagattttggatggcgaaaactgcaccctgatcgacgcgcttctgggggaccctcagtgtgacggctttcagaataagaaatgggatctgtatgtagaacgttcctcggcctatagcaactgctacccgtacgatgtgccggactatgcctcgctccgttcgctggtggcaagcagcgggactctcgaattcaacaacgaatcattcaactgggctggtgttacacaaaatggtacctctagcgcctgcaaacgtcgttccaataaaagctttttttcgcgtttgaattggctcacgcatctgaaatacaagtacccagccttaaacgtaacgatgccgaacaatgagaagtttgacaaactctacgtttggggagtgcaccacccggtcacggattcagaccagatctccctgtatgcccaggccagcggccgtatcacagtcagtaccaagcgctcgcagcaaaccgttatcccgaatatcggctatcgcccgcgcgttcgtgatattagtagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaagatacactggaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 9 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQSSSTGGICDSPHQILDGENCTLIDALLGDPQCDGFQNKKWDLYVERSSAYSNCYPYDVPDYASLRSLVASSGTLEFNNESFNWAGVTQNGTSSACKRRSNKSFFSRLNWLTHLKYKYPALNVTMPNNEKFDKLYVWGVHHPVTDSDQISLYAQASGRITVSTKRSQQTVIPNIGYRPRVRDISSRISIYWTIVKPGDILLINSTGNLIAPRGYFKIRSGKSSIMRSDAPIGKCNSECITPNGSIPNDKPFQNVNRITYGACPRYVKQDTLEAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 10 - DNA, 2049 bp, Artificial Sequence
  HL834 STF2R3 HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaaaactcctctatcggcgaaatctgcgacagcccgcatcagattctggatggcgaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaacaacagcttttttagccgcctgaactggctgacccaactgaactttaaatatccggcgctgaacgtgaccatgccgaacaacgaacagtttgataaactgtatatttggggcgtgcatcatccggtcaccgataaagatcagatttttctgtatgcgcagtcgagcggccgcattaccgtgagcaccaaacgcagccagcaggccgtgatcccgaacattggctaccgcccgcgcattcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaagatacactggaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 11 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTQLNFKYPALNVTMPNNEQFDKLYIWGVHHPVTDKDQIFLYAQSSGRITVSTKRSQQAVIPNIGYRPRIRNIPSRISIYWTIVKPGDILLINSTGNLIAPRGYFKIRSGKSSIMRSDAPIGKCNSECITPNGSIPNDKPFQNVNRITYGACPRYVKQDTLEAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 12 - DNA, 2049 bp, Artificial Sequence
  HL835 STF2R3 HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaaaactcctctatcggcgaaatctgcgacagcccgcatcagattctggatggcgaaaactgcaccctgattgatgcgctgctgggcgatccgcagtgcgatggctttcagaacaaaaaatgggatctgtatgtggaacgcagcagcgcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaacaacagcttttttagccgcctgaactggctgacccaactgaactttaaatatccggcgctgaacgtgaccatgccgaacaacgaacagtttgataaactgtatgtttggggcgtgcatcatccggtcaccgataaagatcagatttttctgtatgcgcagtcgagcggccgcattaccgtgagcaccaaacgcagccagcaggccgtgatcccgaacattggctaccgcccgcgcattcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaagatacactggaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 13 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQNSSIGEICDSPHQILDGENCTLIDALLGDPQCDGFQNKKWDLYVERSSAYSNCYPYDVPDYASLRSLVASSGTLEFNNESFNWTGVTQNGTSSACIRRSNNSFFSRLNWLTQLNFKYPALNVTMPNNEQFDKLYVWGVHHPVTDKDQIFLYAQSSGRITVSTKRSQQAVIPNIGYRPRIRNIPSRISIYWTIVKPGDILLINSTGNLIAPRGYFKIRSGKSSIMRSDAPIGKCNSECITPNGSIPNDKPFQNVNRITYGACPRYVKQDTLEAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 14 - DNA, 2049 bp, Artificial Sequence
  HL836 STF2R3 HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaaaactcctctatcggcgaaatctgcgacagcccgcatcagattctggatggcgaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaacaacagcttttttagccgcctgaactggctgacccaactgaactttaaatatccggcgctgaacgtgaccatgccgaacaacgaacagtttgataaactgtatatttggggcgtgcatcatccggtcaccgataaagatcagatttttctgtatgcgcagtcgagcggccgcattaccgtgagcaccaaacgcagccagcaggccgtgatcccgaacattggctaccgcccgcgcattcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaagcacactgaaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 15 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTQLNFKYPALNVTMPNNEQFDKLYIWGVHHPVTDKDQIFLYAQSSGRITVSTKRSQQAVIPNIGYRPRIRNIPSRISIYWTIVKPGDILLINSTGNLIAPRGYFKIRSGKSSIMRSDAPIGKCNSECITPNGSIPNDKPFQNVNRITYGACPRYVKQSTLKAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 16 - DNA, 2049 bp, Artificial Sequence
  HL899 STF2R3 HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaatcctcctctacgggcggtatctgtgatagcccacaccagattttggatggcgaaaactgcaccctgatcgacgcgcttctgggggaccctcagtgtgacggctttcagaataagaaatgggatctgtttgtagaacgttccaaggcctatagcaactgctacccgtacgatgtgccggactatgcctcgctccgttcgctggtggcaagcagcgggactctcgaattcaacaacgaatcattcaactgggctggtgttacacaaaatggtacctctagcgcctgcaaacgtcgttccaataaaagctttttttcgcgtttgaattggctcacgcatctgaaatacaagtacccagccttaaacgtaacgatgccgaacaatgagaagtttgacaaactctacatttggggagtgcaccacccggtcacggattcagaccagatctccctgtatgcccaggccagcggccgtatcacagtcagtaccaagcgctcgcagcaaaccgttatcccgaatatcggctatcgcccgcgcgttcgtgatattagtagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaagatacactggaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 17 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQSSSTGGICDSPHQILDGENCTLIDALLGDPQCDGFQNKKWDLFVERSKAYSNCYPYDVPDYASLRSLVASSGTLEFNNESFNWAGVTQNGTSSACKRRSNKSFFSRLNWLTHLKYKYPALNVTMPNNEKFDKLYIWGVHHPVTDSDQISLYAQASGRITVSTKRSQQTVIPNIGYRPRVRDISSRISIYWTIVKPGDILLINSTGNLIAPRGYFKIRSGKSSIMRSDAPIGKCNSECITPNGSIPNDKPFQNVNRITYGACPRYVKQDTLEAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 18 - DNA, 2049 bp, Artificial Sequence
  HL747 STF2R3 HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaatcctcctctacgggcggtatctgtgatagcccacaccagattttggatggcgaaaactgcaccctgatcgacgcgcttctgggggaccctcagtgtgacggctttcagaataagaaatgggatctgtttgtagaacgttccaaggcctatagcaactgctacccgtacgatgtgccggactatgcctcgctccgttcgctggtggcaagcagcgggactctcgaattcaacaacgaatcattcaactgggctggtgttacacaaaatggtacctctagcgcctgcaaacgtcgttccaataaaagctttttttcgcgtttgaattggctcacgcatctgaaatacaagtacccagccttaaacgtaacgatgccgaacaatgagaagtttgacaaactctacatttggggagtgcaccacccggtcacggattcagaccagatctccctgtatgcccaggccagcggccgtatcacagtcagtaccaagcgctcgcagcaaaccgttatcccgaatatcggctatcgcccgcgcgttcgtgatattagtagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaaatacactgaaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 19 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQSSSTGGICDSPHQILDGENCTLIDALLGDPQCDGFQNKKWDLFVERSKAYSNCYPYDVPDYASLRSLVASSGTLEFNNESFNWAGVTQNGTSSACKRRSNKSFFSRLNWLTHLKYKYPALNVTMPNNEKFDKLYIWGVHHPVTDSDQISLYAQASGRITVSTKRSQQTVIPNIGYRPRVRDISSRISIYWTIVKPGDILLINSTGNLIAPRGYFKIRSGKSSIMRSDAPIGKCNSECITPNGSIPNDKPFQNVNRITYGACPRYVKQNTLKAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 20 - DNA, 2025 bp, Artificial Sequence
  HL532 STF2R3.HA1-1
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctcaatcctcctctacgggcgaaatctgcgacagcccgcatcagattctggatggcaaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatatttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaaggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 21 - protein, 670 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

SEQ ID NO: 22 - DNA, 2049 bp, Artificial Sequence
  HL533 STF2R3.HA1-1L
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagctgagctggtacaatcctcctctacgggcgaaatctgcgacagcccgcatcagattctggatggcaaaaactgcaccctgattgatgcgctgctgggcgatccgcagtgcgatggctttcagaacaaaaaatgggatctgtttgtggaacgcagcaaagcgtatagcaactgctatccgtatgatgtgccggattatgcgagcctgcgcagcctggtggcgagcagcggcaccctggaatttaacaacgaaagctttaactggaccggcgtgacccagaacggcaccagcagcgcgtgcattcgccgcagcaaaaacagcttttttagccgcctgaactggctgacccatctgaactttaaatatccggcgctgaacgtgaccatgccgaacaacgaacagtttgataaactgtatatttggggcgtgcatcatccgggcaccgataaagatcagatttttctgtatgcgcaggcgagcggccgcattaccgtgagcaccaaacgcagccagcagaccgtgagcccgaacattggcagccgcccgcgcgtgcgcaacattccgagccgcattagcatttattggaccattgtgaaaccgggcgatattctgctgattaacagcaccggcaacctgattgcgccgcgcggctattttaaaattcgcagcggcaaaagcagcattatgcgcagcgatgcaccgatcggcaagtgcaactcagaatgtattaccccgaatggttctatacctaacgacaaacctttccaaaacgtgaaccgcattacctacggtgcctgtcccaggtatgtcaagcaaaatacactgaaagcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttacgttagctcgag

SEQ ID NO: 23 - protein, 678 aa, Artificial Sequence
  Fusion Protein
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAAQVDALRSDLGAVQNRFNSAITNLGNTVNNLSEARSRIEDSDYATEVSNMSRAQILQQAGTSVLAQANQVPQNVLSLLR

(19) United States
(12) Patent Application Publication
(10) Pub. No.: US 2014/0235836 A1
(43) Pub. Date: Aug. 21, 2014

(54) Immunologic Constructs and Methods

(71) Applicant: VaxInnate Corporation, Cranbury, NJ (US)

(72) Inventors: Langzhou Song, Cranbury, NJ (US); Ge Liu, Cranbury, NJ (US); Scott Umlauf, Cranbury, NJ (US); Uma Kavita, Cranbury, NJ (US); Hong Li, Cranbury, NJ (US); Xiangyu Liu, Cranbury, NJ (US); Bruce Weaver, Cranbury, NJ (US); Lynda Tussey, Cranbury, NJ (US)

(73) Assignee: VaxInnate Corporation, Cranbury, NJ (US)

(21) Appl. No.: 14/264,745

(22) Filed: Apr. 29, 2014

Related U.S. Application Data
(63) Continuation of application No. PCT/US2012/000367, filed on Aug. 22, 2012.
(60) Provisional application No. 61/628,739, filed on Nov. 4, 2011.

(51) Int. Cl. C07K 14/47; C07K 14/195

(52) U.S. Cl. CPC C07K 14/47; C07K 14/195
USPC 530/396

(57) ABSTRACT

The present invention relates to improved vaccines and the design and making of such vaccines that enhance immunogenicity of the vaccine and/or reduce reactogenicity to the vaccine when administered. In particular the vaccines and immunogenic compositions of the present invention relate to flagellin-antigen fusion proteins in which the spatial orientation of the flagellin to antigen and the charge distribution of the antigen is optimized to enhance immunogenicity and/or reduce reactogenicity and/or improve folding of the protein.

### RELATED APPLICATIONS

This application is a continuation of International Application No. PCT/US2012/000367, which designated the United States and was filed on Aug. 22, 2012, published in English, which claims the benefit of U.S. Provisional Application No. 61/628,739, filed on Nov. 4, 2011. The entire teachings of the above applications are incorporated herein by reference.

### STATEMENT REGARDING FEDERALLY SPONSORED RESEARCH OR DEVELOPMENT

This invention was made with government support under HHSO01002011000011C from the Biomedical Advanced Research and Development Authority. The government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:

a) File name: 37101053002SeqList.txt; created Apr. 24, 2014, 96 KB in size.

### FIELD OF THE INVENTION

The present invention relates to improved vaccines and the design and making of such vaccines that enhance immunogenicity of the vaccine and/or reduce reactogenicity of the vaccine when administered. In particular, the vaccines and immunogenic compositions of the present invention relate to flagellin-antigen fusion proteins in which the spatial orientation of the flagellin to antigen and the charge distribution of the antigen are optimized to enhance immunogenicity and/or reduce reactogenicity and/or improve refolding of the protein construct.

### BACKGROUND OF THE INVENTION

A number of influenza vaccine formats which fuse subunits of the hemagglutinin (HA) protein to flagellin have been developed. HA is the major protective antigen for influenza and a subunit of HA referred to as HA1-2 appears to be the minimally protective subunit as demonstrated in preclinical lethal challenge models. A longer subunit referred to as HA1-1 genetically fused to flagellin has also been shown to be protective in the preclinical models (see FIG. 1). For certain subtypes of influenza, a genetic fusion of even longer head domains, referred to as HA1-1L and HA1s, attached to flagellin may be more immunogenic than the shorter subunits.

In addition to using different lengths of the HA antigen, vaccine formats which differ in the attachment point of the vaccine antigen to flagellin have also been developed (see FIG. 2). Some formats carry two copies of the antigen. C-terminal format type vaccines genetically fuse the vaccine antigen to the C terminus of flagellin. R3 format vaccines replace domain 3 by genetically fusing the vaccine antigen to flagellin domain D2, R3.2x format vaccines fuse one copy of the vaccine antigen to the C terminus while an additional copy of the antigen replaces domain 3. Each of these different vaccine formats has different properties. More specifically, the attachment point, or location, of the antigen relative to flagellin can influence the antigenic, the immunogenic and even the reactogenic properties of the vaccine.

The different vaccine formats are thought to influence immunogenicity and reactogenicity by modulating the TLR5 agonist properties of flagellin and/or enhancing the display of the vaccine antigen to immune cells. H1 and H5 influenza subtype vaccines of the R3 and R3.2x formats are highly immunogenic, protective in preclinical challenge models and are also tolerated to higher doses than the equally immunogenic but more reactogenic C-terminal formats. For the influenza B and H3 subtypes however, unmodified R3 and R3.2x vaccine formats have proven to be problematic.

Therefore, there is a need to produce vaccines in which influenza antigens are coupled to flagellin that are more immunogenic, less reactogenic and amenable to manufacture at large scale particularly for influenza subtypes other than H1 and H5.

### SUMMARY OF THE INVENTION

The present invention relates to immunological compositions comprising flagellin and at least one antigen in which the length of the antigen, the charge and/or hydrophobicity of the antigen and the orientation of the antigen to the flagellin is altered such that the compositions are more immunogenic and/or less reactogenic.

The present invention describes new vaccines and immunologic compositions in which the relative orientation of the antigen to the flagellin is altered such that the vaccine is more immunogenic and/or less reactogenic.

The present invention describes new vaccines and immunologic compositions in which the relative orientation of the antigen to the flagellin is altered such that the protein construct is more easily refolded and thus more amenable to manufacture.

The present invention describes an immunologic fusion protein comprising flagellin and HA linked together by a linker wherein the length of the linker and charge of the linker is optimized to increase immunogenicity and/or reduce reactogenicity of the fusion protein.

The present invention describes an immunologic fusion protein comprising flagellin and HA linked together by a linker wherein the length of the linker and charge of the linker is optimized to improve refolding and thus improve the ability to manufacture the fusion protein.

The present invention describes a method of improving the antigenicity of flagellin-antigen fusion proteins comprising optimizing the spatial orientation of the antigen to the flagellin by changing the linker length and/or charge such that a TLR5 binding site on the flagellin is not altered.

The present invention describes a method of improving the antigenicity of flagellin-antigen fusion proteins comprising optimizing the charge distribution of the antigen such that a TLR5 binding site on the flagellin is not altered.

The present invention describes a method of improving the antigenicity of flagellin-antigen fusion proteins comprising decreasing the pI of the antigen such that a TLR5 binding site on the flagellin is not altered.

The present invention describes an immunologic composition comprising a flagellin and an antigen wherein the pI of the antigen has been altered such that the pI of the altered antigen is less than the pI of the unaltered antigen.

### BRIEF DESCRIPTION OF THE FIGURES

FIG. 1. A schematic representation of full length hemagglutinin (HA1) and truncated versions of HA, HA1-2, HA1-1, HA1-1L and HA1s.

FIG. 2. A schematic representation of different HA vaccine formats (C-term, R3, R3L, R23 and R3.2x). D0, D1, D2 and D3 are the four domains of flagellin and the genetically fused antigen is encircled. The primary TLR5 binding site is located in D1.

FIG. 3. A diagram illustrating the upregulation of a representative TLR5 responsive gene following immunization with different flagellin based vaccines.

FIG. 4. A schematic representation of the orientation of the globular head domain of HA relative to flagellin in two constructs.

FIGS. 5A-5C. Sequence alignments of multiple H3N2 HA globular heads from virus isolates of human and avian origin were compared. Avian isolates: A/American black duck/Quebec/11235/2006; A/American green-winged teal/Wisconsin/08OS2291/2008; and Ruddy Turnstone/Delaware/SG-00469/2008. Human isolates: Aichi/2/1968; Perth/16/2009; A/Wyoming/03/2003. Key residues, which are different between human and avian isolates but conserved within their own group, are highlighted with boxes.

FIG. 6. A graphical representation of HAI titers of sera from mice immunized with H3N2 Perth candidates.

FIG. 7. Neutralization Inhibition Assay (NIA) data: HA1-1L with globular head and linker substitutions STF2.HA3 (PE) (VAX181 or HL490) competes with virus for binding to neutralizing serum.

FIG. 8A. In vivo TLR5 screen of varying head length of Perth strain H3.

FIG. 8B. A graphical representation of in vivo TLR5 Cytokines for H3 Aichi construct.

FIGS. 9A and 9B. In vivo TLR5 of head length and avian substitutions on Aichi candidates.

FIGS. 10A-D. ELISA titration curves and Biacore binding curves using Aichi specific monoclonal antibodies and a panel of Aichi constructs.

FIG. 11. HAI titers of mouse sera following immunizations with STF2R3.HA3 (AI) wild type or with globular head and linker substitutions.

FIG. 12. HAI titers of R3.HA1L H3 Wyoming (WY) and Victoria strains.

### DEFINITION OF TERMS

“Fusion protein” as used herein refers to a protein generated from at least two distinct components (e.g. a protein portion of HA and a flagellin). Fusion proteins can be generated recombinantly or chemically.

“A portion of a protein” or “protein portion” as used herein in reference to a naturally occurring viral hemagglutinin, refers to any part of the naturally occurring viral hemagglutinin that is less than the entire naturally occurring hemagglutinin.

“A globular head” as that phrase is used herein, refers to a portion of a protein of a naturally occurring viral hemagglutinin that includes the receptor or sialic acid binding regions.

“HA1-1” as used herein, refers to a protein portion of a viral hemagglutinin that includes at least one β-sandwich that includes the substrate binding site, which includes at least about two β sheets, at least about two to about three short α-helixes, at least one small β sheet and at least one additional small β sandwich at the bottom of the molecule and at least about four disulfide bonds. The β sandwich that includes the substrate binding site of the HA1-1 includes about four β-strands as the bottom sheet. At least about one a helix of the HA1-1 portion is located by the side of the β sandwich that includes the substrate binding site and at least about one to about two are located at the bottom of the β sandwich that includes the substrate binding site. The small β sandwich of the HA1-1 can include at least about two to about three β-strands in each β sheet; or about three to about four β-strands. “HA1-1L” as used herein, refers to the extension of the HA1-1 by at least 3 amino acids on the N terminus and 5 amino acids on the C terminus such that the strands form two antiparallel beta strands and then close underneath. In certain embodiments of HA1-1L the number of amino acids added to the N terminus can be greater than three, for example from between 3 and 100 or between 3 and 50 or between 3 and 25 or between 3 and 10. In certain embodiments of HA1-1L the number of amino acids added to the N terminus can be greater than three such as 4, 5, 6, 7, 8, 9 or 10 amino acids added. In certain embodiments of HA1-1L the number of amino acids added to the C terminus can be greater than five, for example from between 5 and 100 or between 5 and 50 or between 5 and 25 or between 5 and 10. In certain embodiments of HA1-1L the number of amino acids added to the N terminus can be greater than five such as 6, 7, 8, 9 or 10 amino acids added.

“HA1-2” as used herein, refers to a protein portion of a viral hemagglutinin that includes at least one β-sandwich that includes the substrate binding site, at least about two to about three short α-helixes, at least one small β sheet at the bottom of the molecule and at least about two disulfide bonds. A β-strand in a viral hemagglutinin can include between about two to about 15 amino acids. A small β-sheet can include from about two to about three β-strands. The β-sandwich that includes the substrate binding site of HA1-2 can further include at least about four β-strands as a top sheet and at least from about three to about four β-strands as the bottom sheet.

### DETAILED DESCRIPTION OF THE INVENTION

Conjugation of flagellin to a vaccine antigen is a way to make a vaccine more immunologically potent and therefore effective. Binding of flagellin to the TLR5 receptor triggers a series of innate and adaptive immune responses that are necessary for orchestration of an effective immune response. A key initial event that follows binding to TLR5 is the propagation of a signal to the nucleus of the immune cell. This signaling event leads to the differential regulation of key genes and the upregulation of cell surface and secreted proteins that are required to initiate an immune response.

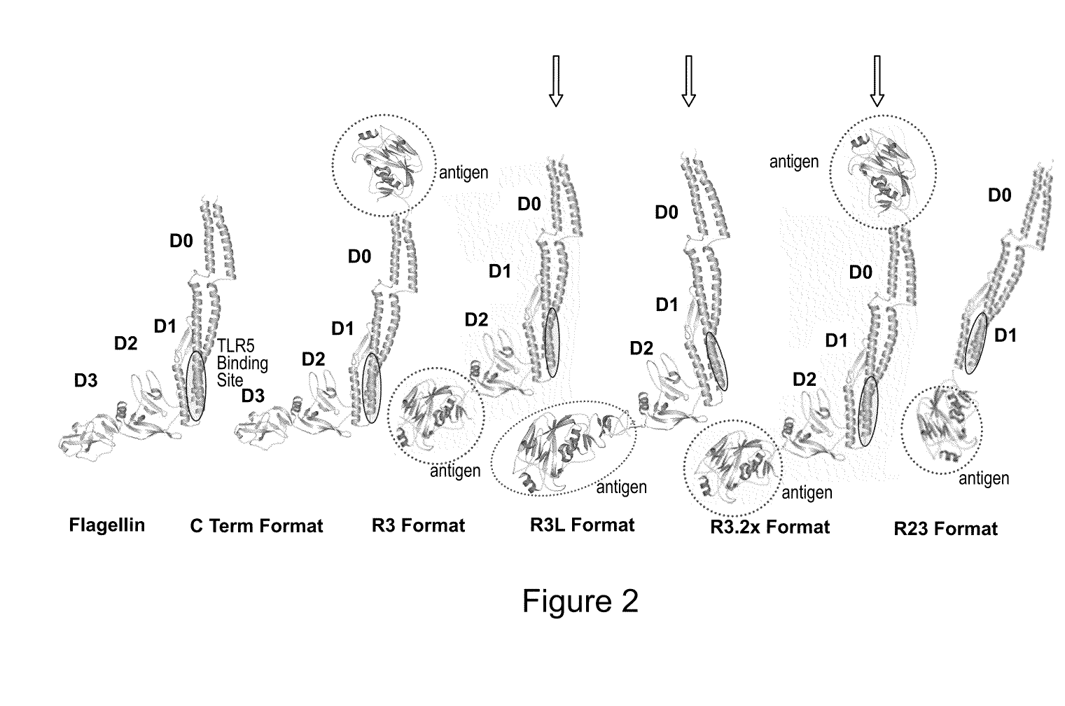

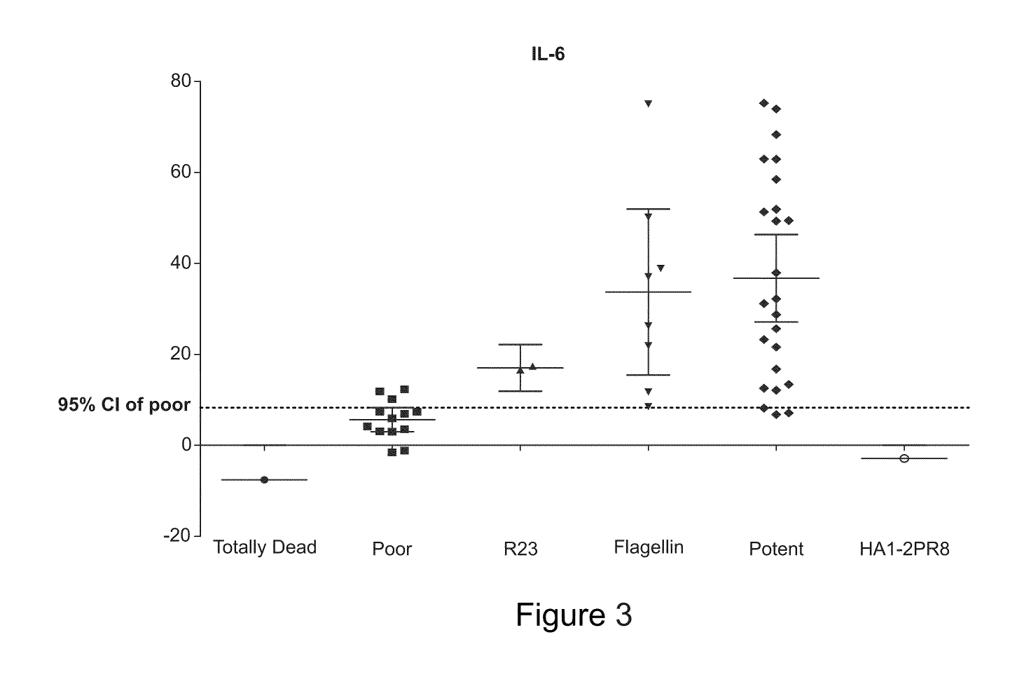

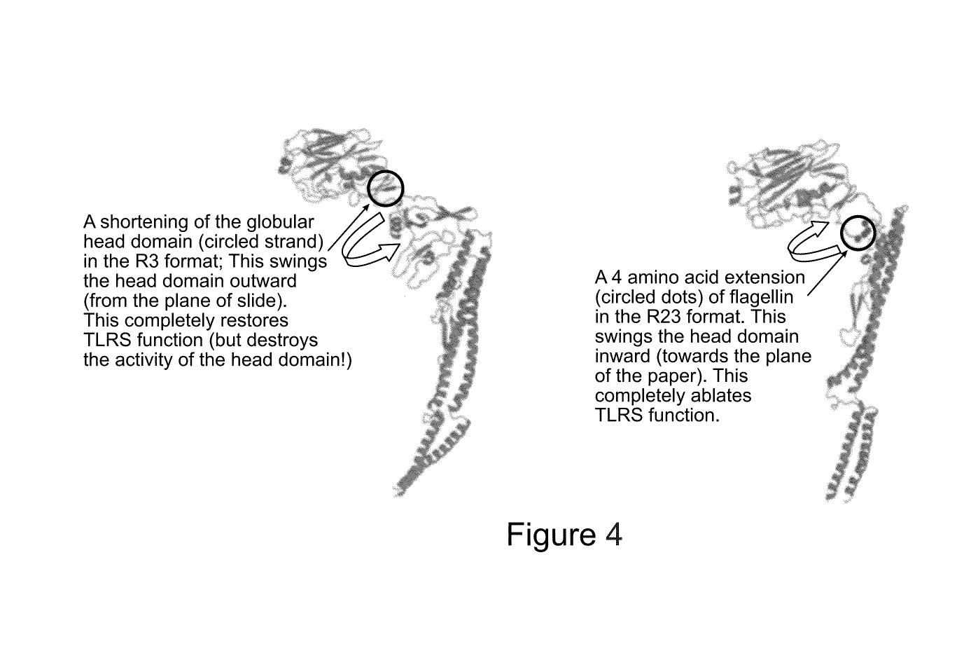

Vaccine compositions utilizing flagellin in combination with one or more antigens which differ in the attachment site of the antigen to flagellin are shown in FIG. 2. Some formats carry two copies of the antigen. C-terminal format type vaccines (C term) genetically fuse the vaccine antigen to the C terminus of flagellin. R3 format vaccines replace domain 3 by genetically fusing the vaccine antigen to D2, R3.2x format vaccines fuse one copy of the vaccine antigen to the C terminus while an additional copy of the antigen replaces domain 3. The fusion proteins comprising a flagellin and at least one antigen can include a linker between at least one component of the fusion protein (flagellin) and at least one other component of the fusion protein (e.g. HA1-1, HA1-2) or any combination thereof. “Linker” as used herein in reference to a fusion protein refers to the connector between components of the fusion protein in a manner that the components are not directly joined. Fusion proteins can include a combination of linker(s) between distinct components of the fusion protein to similar or like components of the fusion protein. The linker can be an amino acid linker which can include naturally occurring or synthetic amino acid residues. The amino acid linker can be of various lengths and compositions. Each of these different vaccine formats has different properties. More specifically, the attachment point, or location, of the antigen relative to flagellin can influence the antigenic, the immunogenic and even the reactogenic properties of the vaccine.

Antigens that can be used in combination with flagellin in the compositions and methods of the present invention are any antigen that will provoke an immune response in a human. Antigens used in the compositions of the present invention include viral antigens such as influenza viral antigens (e.g. hemagglutinin (HA) protein, matrix 2 (M2) protein, neuraminidase), respiratory synctial virus (RSV) antigens (e.g. fusion protein, attachment glycoprotein), papillomaviral (e.g. human papilloma virus (HPV), such as an E6 protein, E7 protein, L1 protein and L2 protein), Herpes Simplex, rabies virus and flavivirus viral antigens (e.g. Dengue viral antigens, West Nile viral antigens), hepatitis viral antigens including antigens from HBV and HC. Antigens used in the compositions of the present invention include bacterial antigens including those from Streptococcus pneumonia, Haemophilus influenza, Staphylococcus aureus, Clostridium difficile and enteric gram-negative pathogens including Escherichia, Salmonella, Shigella, Yersinia, Klebsiella, Pseudomonas, Enterobacter, Serratia, Proteus. Antigens used in the compositions of the present invention include fungal antigens including those from Candida spp., Aspergillus spp., Crytococcus neoformans, Coccidiodes spp., Histoplasma capsulatum, Pneumocystis carinii, Paracoccidiodes brasiliensis, Plasmodium falciparum, Plasmodium vivax, Plasmodium ovale, and Plasmodium malariae. In preferred embodiments the antigen contained within the compositions of the present invention is an antigen from influenza virus. A preferred antigen is hemagglutinin (HA). In preferred embodiments the HA sequences are conjugated to flagellin or to engineered flagellins as described in WO 2009/128950 herein incorporated by reference.

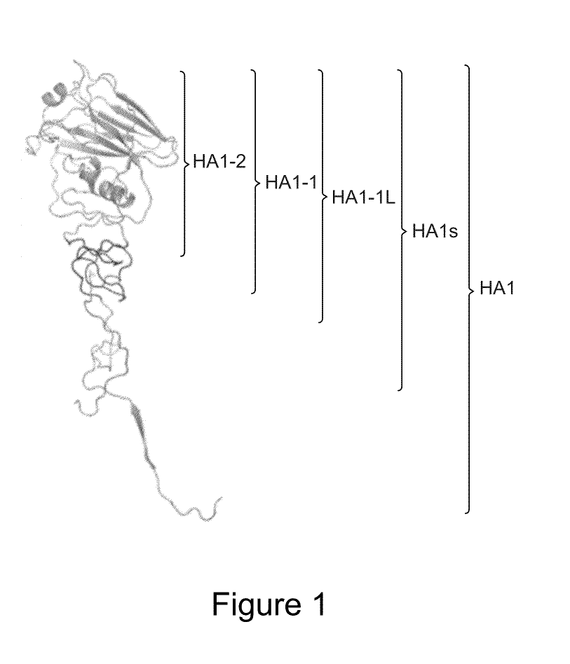

FIG. 1 shows a ribbon diagram of an HA antigen from influenza. HA1 is the full-length HA1 subunit of hemagglutinin. HA1-1, HA1-2, HA1-1L and HA1-s are truncated versions of the HA1 subunit. Genetic fusions of the HA1-2 or HA1-1 HA subunit of influenza virus have been constructed for several different subtypes of influenza. HA1-1 and HA1-2 are further described in WO 2007/103322, herein incorporated by reference. HA1-1 and HA1-2 subunits from multiple HAs of the H1 and H5 subtypes to flagellin are highly immunogenic and efficacious as demonstrated in preclinical challenge models. For influenza B however, when these subunits are presented in a C term or R3 vaccine format, the vaccines are poorly immunogenic. Specifically, C term forms of influenza B Florida HA have not elicited protective immune responses in naïve mice nor do they boost pre-existing titers in primed mice. R3 forms of influenza B Florida vaccines only elicit measurable immune responses in animals that have already received an immunization of commercial vaccine (primed animals). The disulfide bonds of all construct formats are properly formed and the antigen reacts well with antibodies specific for the antigen, indicating that the antigen has folded properly. Thus, the lack of activity appears not to be related to improper folding of the HA antigen.

There are a set of genes and cytokine proteins, along with thresholds for their production, that are important for the immune potentiation function of flagellin. Measurement of these genes or proteins can be used as guides to design more immunogenic vaccines.

Multiple HAs of the H3 subtype genetic fusions of the HA1-2 HA subunit presented in the standard flagellin formats are poorly immunogenic. Specifically, C term and R3 forms of H3 Wisconsin, H3 Aichi and H3 Perth HA1-2 HA fail to elicit protective immune responses in naïve mice or boost pre-existing titers in primed mice. Moreover, H3 vaccines utilizing the HA1-2 length of globular head have a tendency to misfold and aggregate during production. In the instances when monomeric protein preparations were produced, these proteins proved to be poor triggers of TLR5 in in vivo based assays and consequently, were poorly immunogenic. Key contributors to the poor immunogenicity appear to include: 1) H3 HAs, similar to influenza B HAs, have a high pI associated with the globular head. This could interfere with TLR5 signaling or promote an unwanted interaction between the HA head and flagellin which has a low pI and/or 2) the human H3 HA globular head has an extremely hydrophic core, as compared to the generally more active H1 and H5 HA globular heads. This could complicate proper refolding of the molecule and make manufacturing of the genetic fusion proteins difficult. By changing the charge or polarity of the amino acid sequence on the surface of the globular head the negative effects of the high pI may be ameliorated. Furthermore, altering the amino acid sequence in the linker region to change the spatial orientation of the globular head may provide an antigen that mimics other antigens that are more highly antigenic. Also, the use of a globular head domain that is longer (in one embodiment approximately 57 amino acids longer than HA1-2 shown as HA1-1L in FIG. 1) provides additional secondary structure underneath the globular head and its use substantially improves H3 production, presumably by facilitating stable refolding of the H3 HA head.

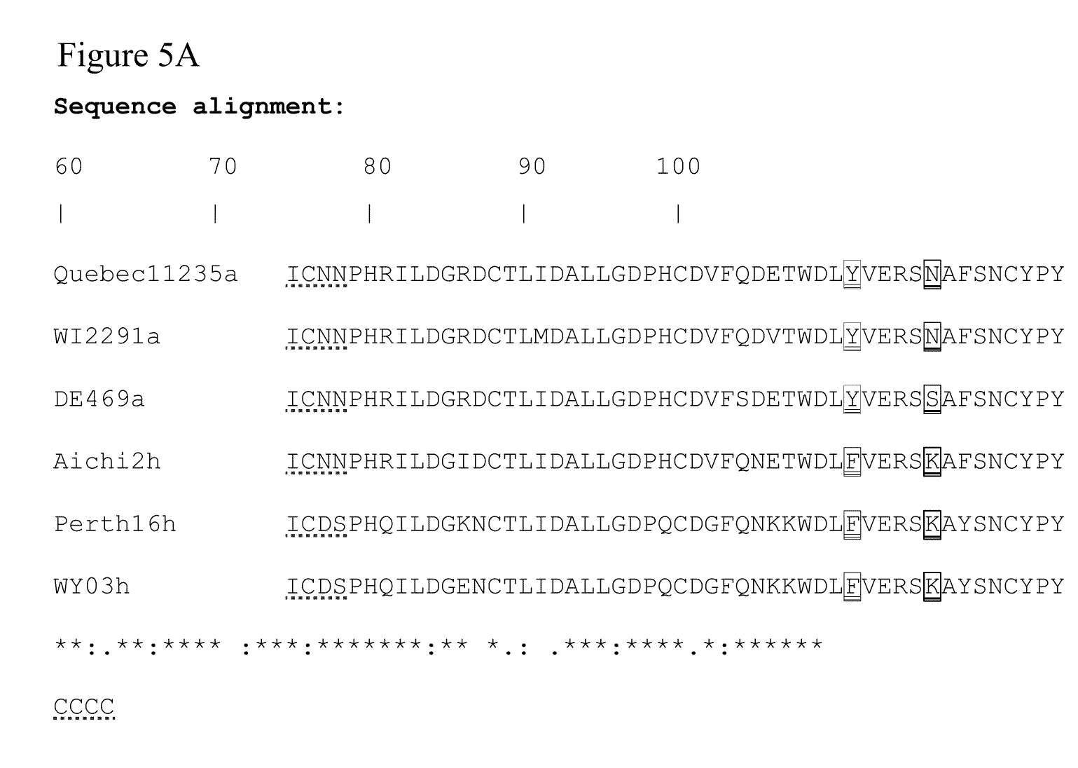

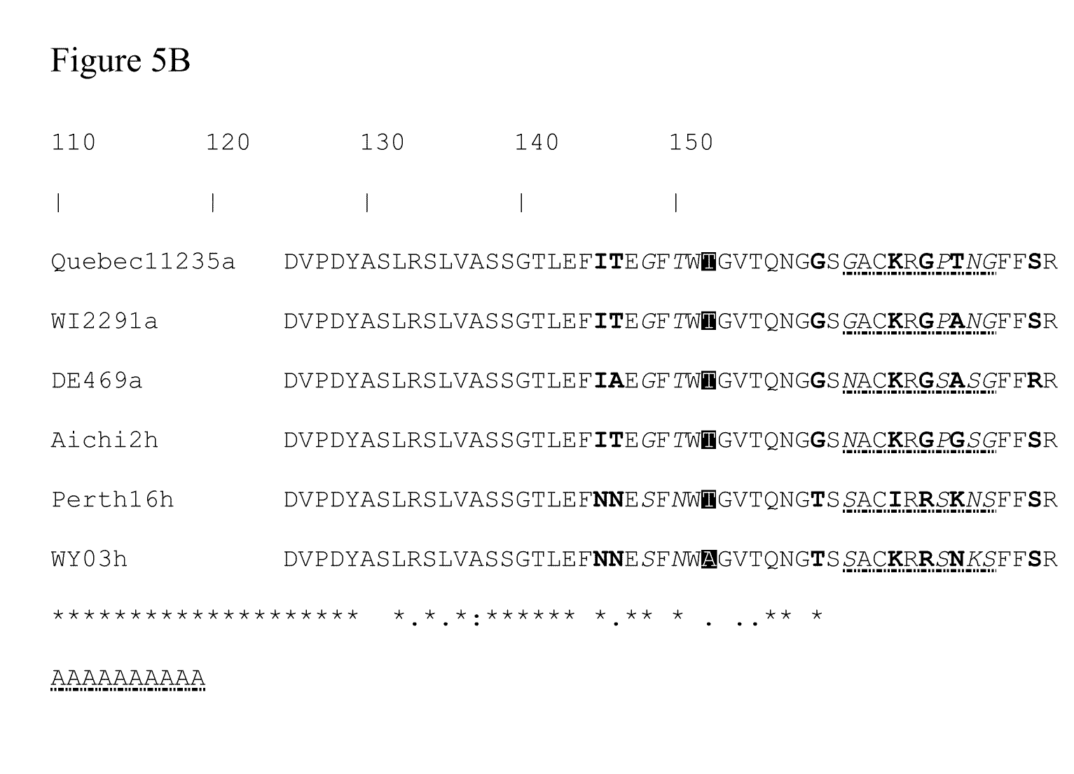

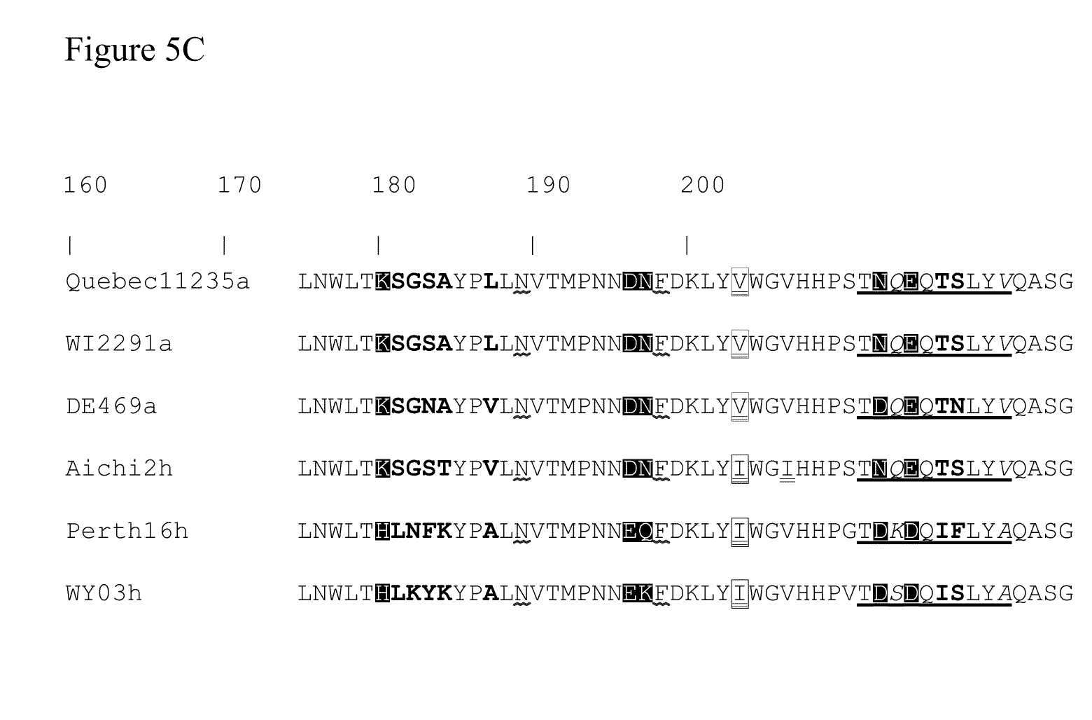

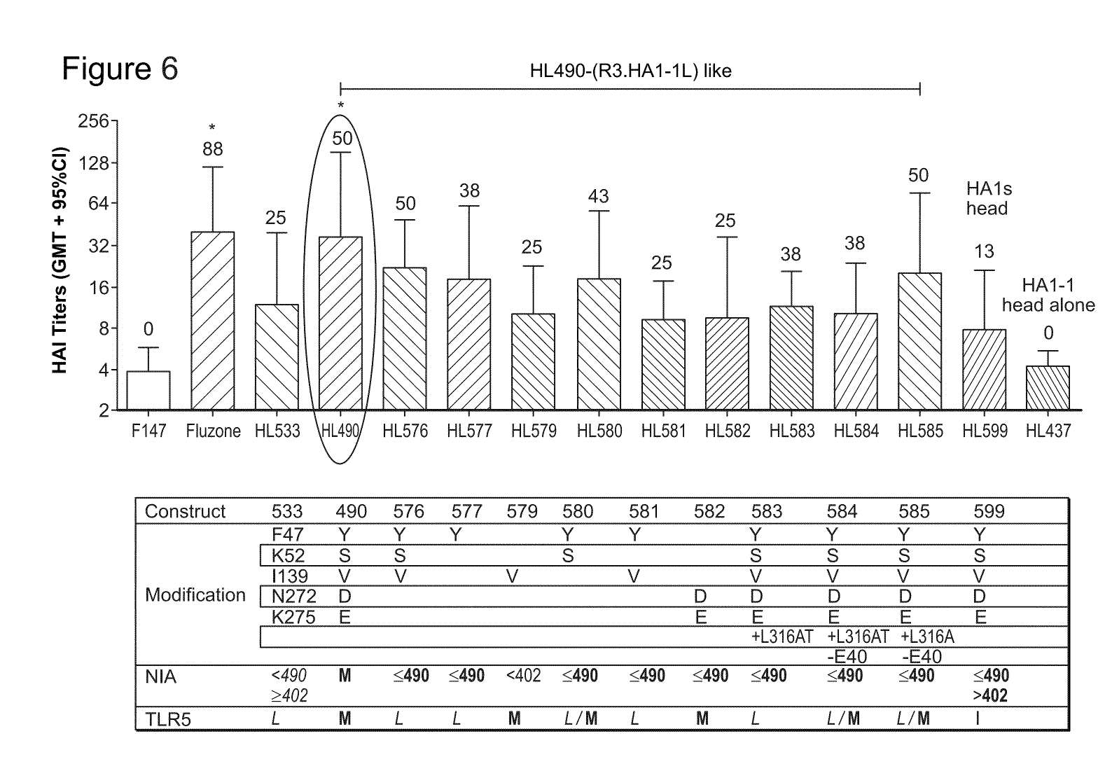

Standard R3 formats with HA1-2 globular heads of H3 vaccine candidates have a tendency to aggregate during the refolding stage of protein purification. Comparisons of HA subtypes indicate that H3 HAs have more hydrophobic amino acids in the core residues that are buried in the interior of the head domain than do H1, H5 or B HAs. Additionally, H3N2 viruses of avian origin (e.g. A/Ruddy Turnstone/Delaware/SG-00469/2008) had less of a tendency to aggregate. A comparison of sequences of human and avian H3 viruses (see FIG. 5) identify three amino acids which differ between human and avian isolates but which are highly conserved within each group. Two of these are buried in the core of HA, and are less hydrophobic in avian isolates than human, while the third residue is on the surface but is situated outside of the major antigenic regions of H3 subtype. Substitution of the human residues with their avian counterparts facilitates production. Together these three changes are referred to as globular head substitutions.

Two H3N2 human isolates, A/Perth/16/2009, which has been recommended for the 2009-2010 seasonal vaccine, and A/Aichi/2/1968 which is one of the most heavily studied influenza strains were tested using the globular head substitutions in these strains. Refolding of the modified strains was significantly better than with wild type sequence, as summarized in Table 1. The avian substitutions in these strains also improved overall recovery of final product.

TABLE 3.A.1.
Refolding Yields of H3 Perth and H3 Aichi HA1-2 Constructs

| Construct Description | Refolding Yield* |
|---|---|
| Perth R3.HA1-2: WT | 2.3% |
| Perth R3.HA1-2: all globular head substitutions | 6.25% |
| Aichi R3.HA1-2: WT | 3% |
| Aichi R3.HA1-2: all globular head substitutions | 6.2% |

*Refolding efficiency was measured by the recovery of folded monomers captured by the subsequent anion exchange chromatography compared to the load material.

Thus, it appears that ‘structural information’ contained within the longer HA1-1L globular head influences the stable refolding of H3 HAs. Substitution of 2 conserved residues in the core and 1 on the surface of HA with the avian counterpart appear to assist stable refolding of the head by alleviating the extreme hydrophobicity of the core and altering the charge of the surface. ELISA, Biacore and melting temperature data support these conclusions (See Examples). Addition of negative charge to the linking region improves potency, possibly by alleviating intramolecular interactions between the positively charged H3 HA head and negatively charged flagellin.

For the higher pI HAs, including most influenza B and H3 antigens, changes in the length and charge content of the region linking the HA head to flagellin have improved the immunogenicity of these vaccines. H3 based vaccines benefit from a longer globular head. Further, substitution of residues in the core and on the surface of the head domain with their avian counterparts has been found to significantly improve the immunogenicity. The inclusion of two negatively charged residues in the region linking HA to flagellin also facilitates production of the H3 fusion proteins. In the case of H3 extension of HA1-1 length to include small anti-parallel β strands provided a substantial positive effect on refolding recoveries as summarized in Table 2. Combining the HA1-1L head with the globular head and linker substitutions yielded the highest efficiencies of all, indicating that the effects of both the head length and substitutions are additive or synergistic. The constructs of the present invention provide an improved re-folding yield such that the yield of construct proteins is increase by greater than 10% as compared to wild type or non-modified constructs. In some embodiments of the constructs of the invention the re-folding yield are between from about 10 to about 1000% higher or from about 10 to about 500% higher or from about 10 to about 100% or from about 10 to about 50% or from about 10 to about 25% higher than wild type or non-modified constructs.

TABLE 2
Refolding Yields of H3 Perth HA1-2 and HA1-1L Constructs

| Construct | | |
|---|---|---|
| Code | Construct Description | Refolding Yield* |
| HL387 | Perth R3.HA1-2: WT | 2.3% |
| HL533 | Perth R3.HA1-1L: WT | 7.1% |
| HL490 | Perth R3.HA1-1L: globular | 23% |
| | head and linker substitutions | |
| HL337 | Aichi R3.HA1-2 : WT | 3% |
| HL700 | Aichi R3.HA1-1L: WT | 10.6% |
| HL792 | Aichi R3.HA1-1L: globular | 9.7% |
| | head substitutions | |
| HL566 | Aichi R3.HA1-1L: globular | 26.1% |
| | head and linker substitutions | |

*Refolding efficiency was measured by the recovery of folded monomers captured by the subsequent anion exchange chromatography compared to the load material.

For example, one embodiment of the present invention is HL490 (SEQ ID 1, 2) which is based on an H3 Perth strain which utilizes the R3L format. In addition to the longer HA1-1L globular head, the HL490 construct contains 5 amino acid modifications. Two of the modifications introduce negative charge in the linking region (in the HA1-1L construct, asparagine is changed to aspartic acid at residue 272 and glutamine is changed to glutamic acid at residue 275), two are modifications to core residues within the interior of the HA head domain (in the HA1-1L construct, phenylalanine is changed to tyrosine at residue 47 and isoleucine is changed to valine at residue 139) and one modification is to a surface residue of the HA head domain in the HA1-1L construct (lysine to serine at residue 52). As can be seen in SEQ ID 2 the numbering for the residues described above begins at line 3 of the sequence with the sequence beginning with ELVQ. The numbering of the residues in other constructs might be different depending on the number of total residues in the HA1-1L construct but the purpose of the substitutions and sites selected for substitution is in keeping with that described herein. The changes to the core and surface are at residues that are highly conserved within avian or human H3 HAs but not across avian and human HAs. In general it is easier to process avian H3 HAs than human H3 HAs. The extreme hydrophobicity of the human core and the higher surface charge associated with human H3 HAs appears to cause human H3 HAs to aggregate. Thus, substitution of these 3 residues with the avian counterpart in human H3 HAs provides compositions which tend not to aggregate. HL490 modifications have also been introduced to the well-studied historical strain Aichi/2/68. HL566 (SEQ ID 6, 7) is a construct which has the HA1-1L head length, the avian substitutions and the negatively charged linker associated with HL490. HL566 demonstrates both IL-6 and TNF in mouse serum are equal to or greater than HL185, which is an R3 H1 vaccine which was shown to be effective in the clinic (VAX128B) and is the target for cytokine expression. Further, innate mRNA upregulation was seen in 10 of 11 genes in screens (vs. 9 of 11 for HL490 and 11 of 11 for HL185 in the same study). HL566 and HL490, with the HA1-1L head, 3 avian substitutions and the negatively charged linker optimize the performance of Aichi vaccines as well as Perth vaccines. These substitutions improve the ease of processing for both Aichi and Perth strains and for Aichi strains also improved the effectiveness of the vaccine in a lethal challenge study. Another construct, HL615 (SEQ ID 8, 9) an R3HA1-1L (HL490-like) version of a recent strain of H3, Wyoming/03/2003, also provides improved antigenicity. With respect to each of the following constructs the pI of the molecule was changed as follows: HL490 wild type, pI 7.95 to 6.30, HL565 wild type, pI 6.01 to 5.74, HL566 wild type, pI 6.09 to 5.53 and HL615 wild-type, pI 7.94 to 6.31. In certain embodiments of the constructs of the present invention the pI of the construct is altered with respect to the wild type such that the pI is decreased by between about 0.1 to about 3.0 units, or from about 0.1 to about 2.0 units or from about 0.1 to about 1.5 units or from about 0.1 to about 1.0 or from about 0.1 to about 0.5 units.

Substitution of 3 conserved residues in the core and on the surface of HA with the avian counterpart appears to assist stable refolding of the head by alleviating the extreme hydrophobicity of the core and the altering the charge of the surface. Addition of negative charge to the linking region improves potency, possibly by alleviating intramolecular interactions between the positively charged H3 HA head and negatively charged flagellin.

The constructs of the present invention include sequences where amino acid modifications reduce the deleterious interactions of the antigen with the flagellin and or promote the folding of the molecule. Suitable amino acid sequence modifications include substitutional, insertional, deletional or other changes to the amino acids of any of the polypeptides discussed herein. Substitutions, deletions, insertions or any combination thereof may be combined in a single variant so long as the variant is an immunogenic polypeptide. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, but are not limited to, M13 primer mutagenesis and PCR mutagenesis. Amino acid substitutions are typically of single residues, but can occur at a number of different locations at once. As described above two amino acid modifications were used to introduce negative charge in the linking region, two were modifications to core residues within the interior of the HA head domain and one modification was to a surface residue. Once the amino acids that can be substituted are identified other modifications may be made at those sites as well. For example, HL 490 might be modified such that asparagine is changed to glutamic acid at residue 272 of HA1-1L and glutamine is changed to aspartic acid at residue 275 of HA1-1L. Such substitutions generally are made in accordance with the following Table and are referred to as conservative substitutions and generally have little or no effect on the size, polarity, charge, hydrophobicity, or hydrophilicity of the amino acid residue at that position and, in particular, does not result in decreased immunogenicity. However, others are well known to those of skill in the art.

TABLE

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Thus, it is now possible on the basis of this idealized charge distribution and appropriate spatial orientation it is now possible to design flagellin-antigen fusions that take these issues into account and permit the rational design of a fusion that is spatially and electrostatically “correct”. That is, gives the optimal charge and spatial orientation between the antigen and flagellin such that antigenicity is increased and/or reactogenicity is decreased and/or folding or refolding of the resultant construct is improved.

### EXAMPLES

### Example 1

### Modified Constructs Using Perth Strains

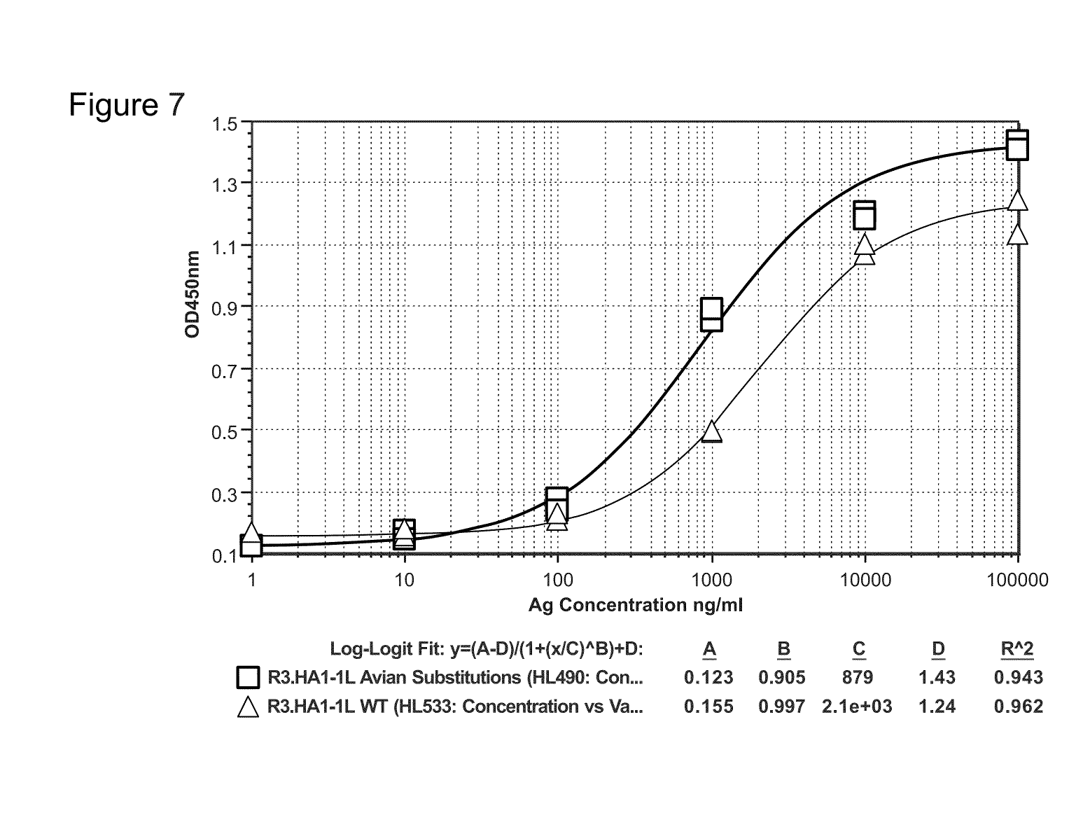

Groups of 8 BALB/c mice were immunized s.c. with indicated candidates at 5 μg dose or Fluzone at 15 μg on days 0 and 21, and bled on day 35. Serum samples were subjected to HAI test using A/Perth/16/09 virus. Data represent geometric mean titers (GMTs) with 95% confident intervals (95% CIs). Seroconversion rates (% mice shows 4-fold raise in HAI titers) are given above each group. *, p<0.05 in Kruskal-Walis/Dunn's tests vs F147 group. Modifications in amino acid residues in single-letter code and corresponding positions are given in FIG. 7. HL533 contains the wild type HA sequence. +L316AT, addition of Leucine316 Alanine317 Threonine318 on the C-terminus of HA head. −E40, deletion of E (Glutamic acid) at position 40. TLR5 activities indicated by serum levels of IL6/TNFα are provided. I, inactive (grey); L, low (green); M, medium (yellow); H, high (red).

Constructs were designed, produced and evaluated in the mouse model of immunogenicity and were also evaluated for TLR5 function and antigenicity. The screen is designed to evaluate the innate stimulating properties as well as the epitope integrity of our candidate vaccines using an in vivo TLR5 bioactivity and a neutralization of inhibition (NIA) assay respectively. The results are summarized in FIG. 7.

The neutralizing antibody results show that HL490 elicited a GMT HAI titer (GMT=37 with 50% seroconversion) comparable to a commercial flu vaccine Fluzone (GMT=40 with 88% seroconversion). Both HL490 and Fluzone induced significant levels of virus neutralizing antibodies (p<0.05 vs. F147 group). Constructs with fewer modifications elicited lower HAI titers. For example:
The construct which contains wild-type sequence of the HA head (amino acids 41-315; HL533), showed only ⅓ HAI titer (GMT=12 with 25% seroconversion) as compared to HL490.
The construct with a single amino acid substitution I139V (HL579) maintained good TLR5 activity, but reduced neutralizing activity of the HA head.
The construct containing three avian-like modifications (F47Y, K52S, and I139V; HL576) elicited a slightly lower level of HAI titers (GMT=22 with 50% seroconversion).
The construct containing two of three avian-like modifications (F47Y & K52S; HL580) is comparable to the construct containing all three modifications (HL576) in GMT HAI titers and seroconversion rates.
Like HL579, the construct containing 2 negatively charged modifications to the linker (N272D & K275E; HL352), showed a HL490 equivalent TLR5 activity, slightly lower NIA activity, and a lower GMT (=10) HAI titer.

These results indicate that the combination of 3 avian-like modifications and 2 negatively charged mutations produces full immunogenicity of HL490. Among the subset mutations, 2 avian-like (F47Y and K52S) mutations might have accounted for improved conformation of the HA head in HL490; whereas the two negatively charged AA (N272D and K275E) mutations in the linker and one avian like mutation (I139V) appear to improve the TLR5 activity as compared to the vaccine construct without modification (HL533). Addition and/or deletion of N-terminus or C-terminus residues (HL582, HL583, HL584, & HL585) did not enhance the immunogenicity. Another R3L Perth candidate (HL599), which contains the same five HL490 like modifications in the longer HA1s head shows no TLR5 activity, resulting in a much lower GMT (=8) HAI titer. As expected, the HA head alone construct (HL437) failed to elicit a significant HAI titer.

The effect of HA head length and globular head and linker substitutions were tested using the Perth strain by first examining the antigenicity using an inhibition of neutralization assay (NIA). NIA is used to measure the ability of a vaccine candidate to bind and deplete neutralizing antibodies present in sheep hyperimmune serum used for the potency assay (SRID) of a commercial influenza vaccine (TIV). This assay therefore, assesses the integrity of neutralizing epitopes on the flagellin-HA fusion immunogen. It should be noted that the underlying elements of this assay are identical to those of the micro-neutralization (MN) test (Rowe et al, 1999), or more specifically neutralization of virus infection of MDCK cells. In the NIA test, the sheep hyperimmune serum raised against a specific influenza virus or its HA antigen (CBER or NIBSC) is pre-incubated with serially diluted antigens for 1.5 hour. Influenza virus is then added and allowed to incubate for 1 hour at 37° C. prior to addition of MDCK cells. Following a 20-hour incubation at 37° C., the cells are fixed. Intracellular influenza virus is quantified by ELISA using NP-specific mAbs as primary antibodies. Curves are fit with a 4-parameter logistic equation (Softmax 5.2, Molecular Devices).

Antigens in duplicate were serially diluted, pre-incubated with sheep hyperimmune reference serum to HA antigen A/Perth/16/2009 obtained from CBER, and then with of A/Perth/16/2009 virus. After overnight infection in MDCK cells at 37° C., replicated virus was detected by ELISA with NP-specific NP MAb and goat anti-mouse IgG:HRP antibody. OD₄₅₀ values are fit with a 4-parameter logistic equation.

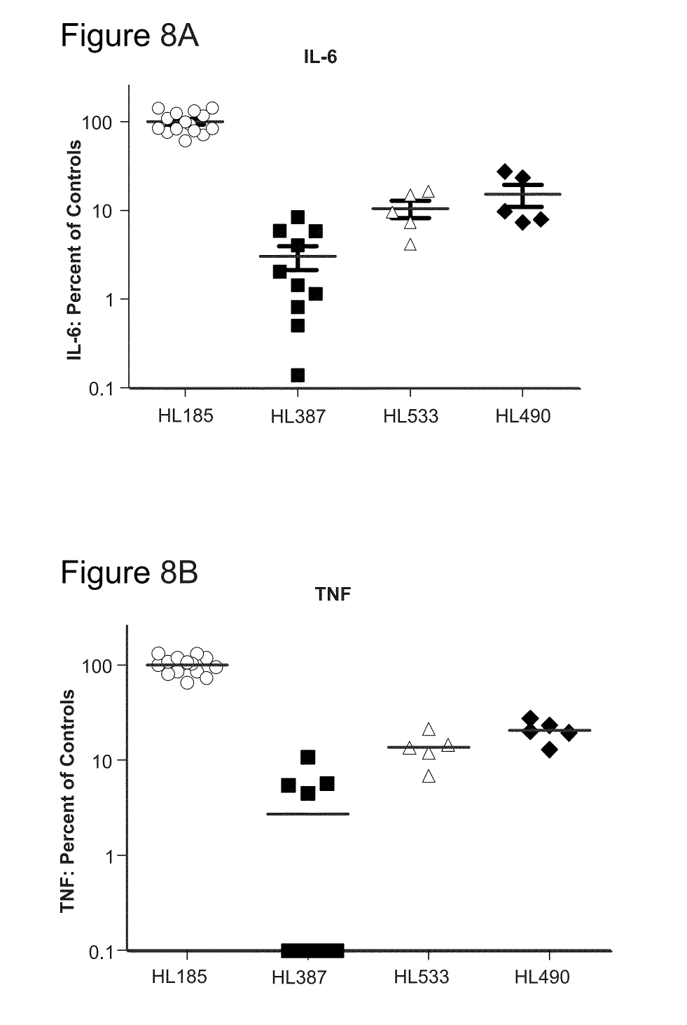

As shown in FIG. 7, both wild type and substituted HA1-1L constructs are highly active in competing for binding to neutralizing antibodies in the NIA assay. In this case the addition of globular head and linker substitutions provides an advantage relative to wild type, presumably by facilitating refolding of the globular head domain. In vivo TLR5 tests were performed in groups of 5 BALB/c mice, immunized with 1 μg of each construct. R3 H1 CA07 (HL185) was included as a positive control; naïve mice were included as negative controls. Serum TNF and IL-6 levels were measured 3 hours after s.c. immunization with the indicated constructs. In order to make comparisons across multiple studies, cytokine levels were converted to percent by comparison to the positive and negative controls. Cytokine levels (percent) of individual mice are shown along with a bar for the group means and standard errors of the mean. FIG. 8: Panel A: IL-6 levels Panel B: TNF-α levels Perth constructs were tested in the mouse in vivo TLR5 model. For both IL-6 and TNF secretion, the wild type HA1-2 sequence (HL387) has very poor activity. As with Aichi, the increase in head length to HA1-1L causes a significant increase in cytokine release for both wild type (HL533) and substituted (HL490) forms. The avian substitutions incrementally increase activity relative to wild type.

### Example 2

### Modified Constructs Using Aichi Strains

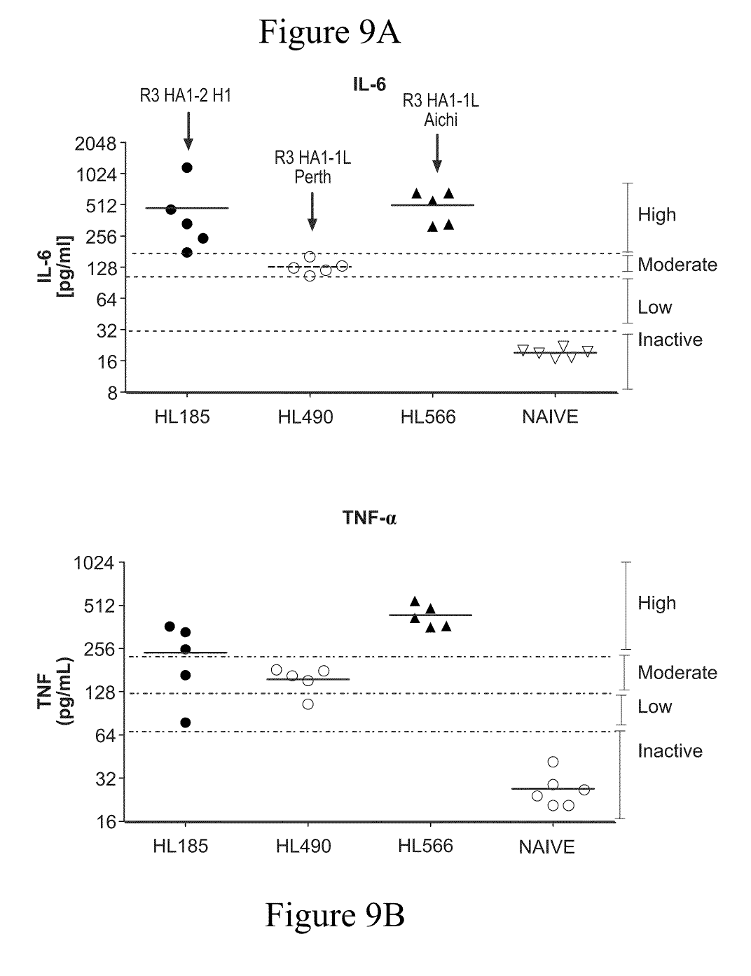

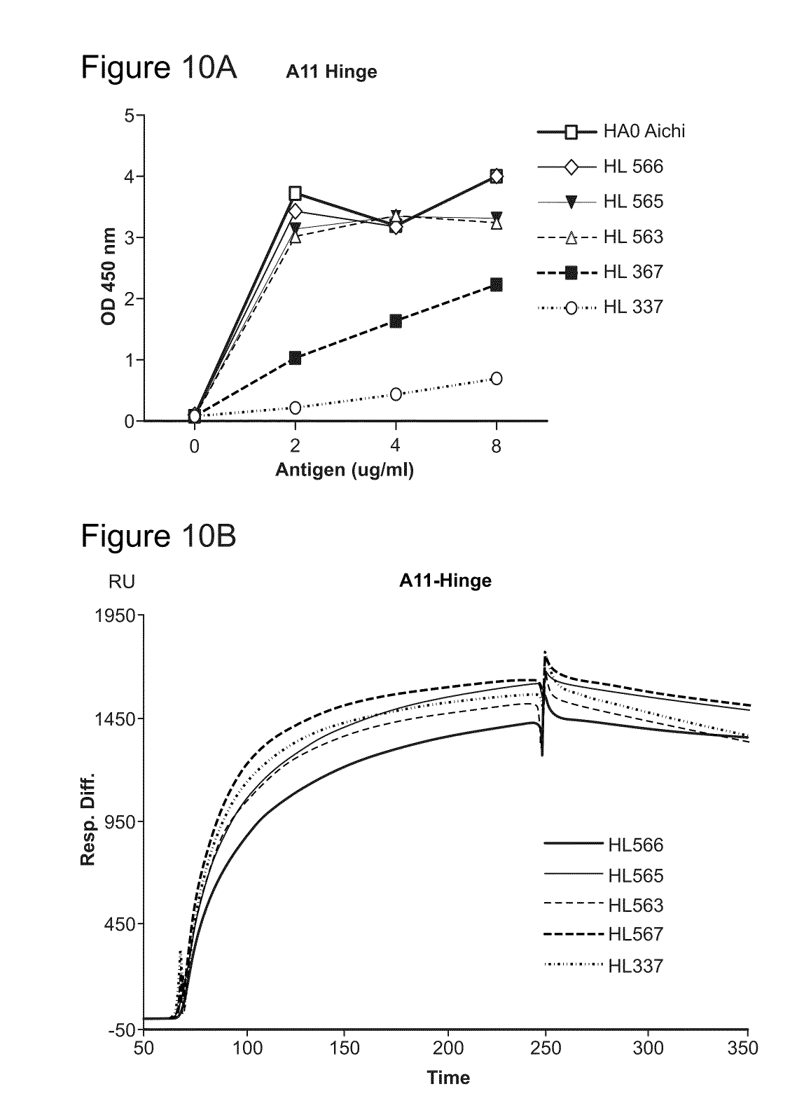

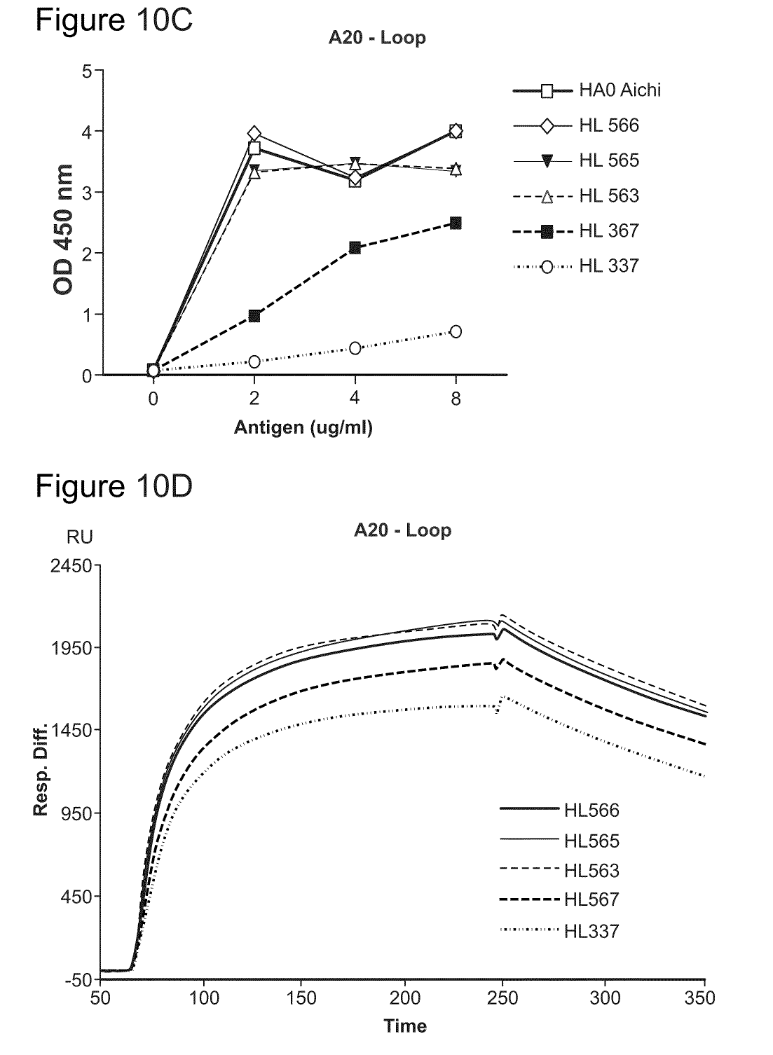

BALB/c mice were injected with 1 μg of each vaccine candidate or left naïve. At 3 hours, mice were bled to generate serum. Cytokine levels were quantified using a mouse inflammation cytometric bead array (BD). IL-6 data is displayed in the top panel and TNF data in the bottom panel of FIG. 9. Vaccines that produce higher IL-6 and TNF expression are considered more active. HL185 (STF2R3.HA1 CA07), HL490 (STF2R3.HA1-1L Perth) are included as positive controls. HL185 results can be regarded as the level of TLR5 triggering that we are targeting with our vaccine optimizations.

TABLE 1
Aichi vaccine candidates with HL490 modifications

| | | Avian | Incor- | | |
|---|---|---|---|---|---|
| | | Substi- | poration | | |
| | Position & | tutions | of Negative | PCR | |
| | Globular | to Core | Amino | (genes | |
| Construct | Head | and | Acids | upreg- | |
| Name | Length | Surface | in Linker | ulated) | Cytokines |
| HL337 | R3; HA1-2 | no | no | 2 of 11 | IL-6 and TNF |
| | | | | genes | low/inactive |
| HL367 | R3; HA1-2 | yes | no | 5 of 11 | IL-6 and TNF |
| | | | | genes | inactive |
| HL563 | R3; HA1s | no | no | 8 of 11 | IL-6 and TNF |
| | | | | genes | low |
| HL565 | R3; HA1s | no | yes | 4 of 11 | IL-6 and TNF |
| | | | | genes | low |
| HL566 | R3; HA1-1L | yes | yes | 10 of 11 | IL-6 and TNF |
| | | | | genes | high |

Five Aichi constructs listed in Table 1 which differ in the length of the HA head, incorporation of subsets of the avian substitutions, and negatively charged residues within the linkers were evaluated for their ability to bind 2 different neutralizing monoclonal antibodies in the ELISA (panel A) and Biacore (panel B) assays. The A11 antibody binds an epitope in the hinge region of the HA head (below the main antigenic regions) while the A20 antibody binds on the top of the head in one of the main antigenic sites. Controls included HA0 Aichi (made by VaxInnate using a baculovirus system). ELISA data are presented as OD 450 nm vs. antigen concentration. Biacore data are presented as individual traces of the indicated vaccine passed over antibody-coated chips. In general, those constructs with the longer globular head domains and the avian substitutions bind these antibodies better, with higher affinity and slower off rates than molecules not carrying these modifications. Titration curves for the ELISA and an example BIAcore binding assay are shown in FIG. 109 AD.

A series of three separate experiments the Aichi constructs were analyzed for their ability to elicit HAI titers in the mouse model. The data, summarized in Table 2, are consistent with the ELISA, BiaCore™ and in vivo TLR5 assays in that HL566 which incorporates the longer head domain, the avian substitutions and the negative charges in the linker is the most active construct. A similar pattern was observed for the H3 Perth vaccine.

TABLE 2
Summary of GMTs and Seroconversion rates for Aichi Constructs

| | | Avian | Incor- | | |
|---|---|---|---|---|---|
| | | Substi- | poration | | |
| | | tutions | of Negative | | |
| | Position & | to Core | Amino | | Sero- |
| Construct | Globular | and | Acids | | conversion |
| Name | Head Length | Surface | in Linker | GMT | Rate (%) |
| F147 | | | | 2.5 | 0 |
| HL337 | R3; HA1-2 | no | no | 2.5 | 0 |
| HL367 | R3; HA1-2 | yes | no | 2.5 | 0 |
| HL563 | R3; HA1s | no | no | 10 | 33 |
| HL565 | R3; HA1s | no | yes | 25 | 50 |
| HL566 | R3; HA1-1L | yes | yes | 36 | 83 |

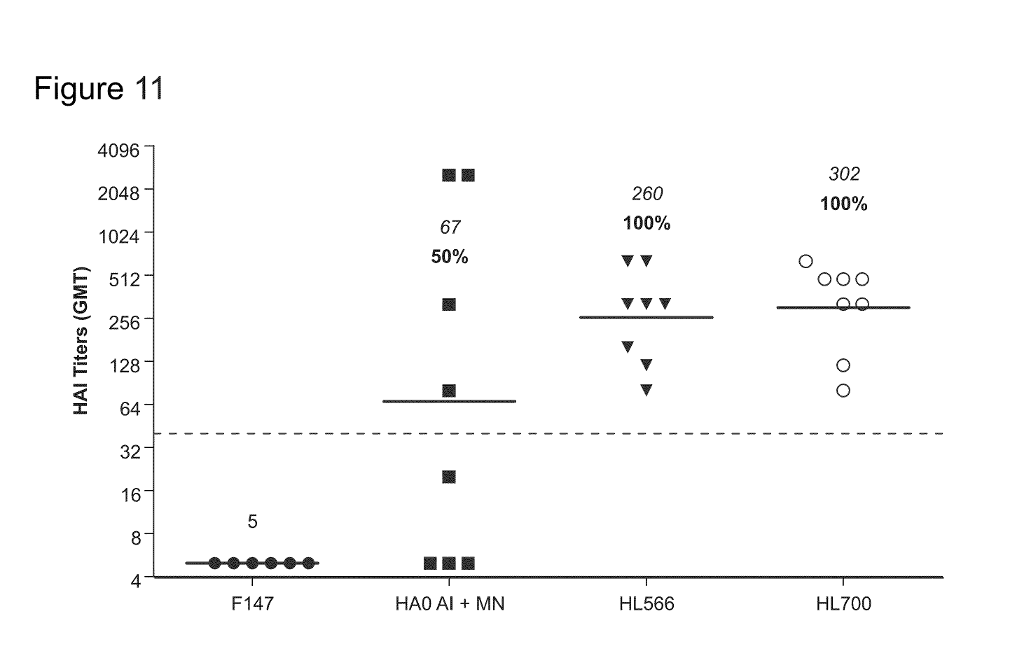

Finally, in order to confirm that the modifications improved the immunogenicity of the Aichi constructs we immunized mice with both wild type and avian substituted vaccines and evaluated the immune sera for HAI titers. Groups of 8 BALB/c mice immunized IM with 6 μg of STF2R3.HA3 (AI) wild type (HL700) or with globular head and linker substitutions (HL566). Controls included baculovirus-produced HA0 Aichi in Montanide adjuvant (HA0 AI+MN) or formulation buffer (F147) on days 0 and 21, and bled on day 35. Serum HAI antibodies were measured by HAI test using A/Aichi/2/1968 virus, and plotted individually. Horizontal lines represent GMTs (green). Seroconversion rate are also given (red) (FIG. 11).

### Example 3

### Modified Constructs Using Wyoming and Victoria Strains

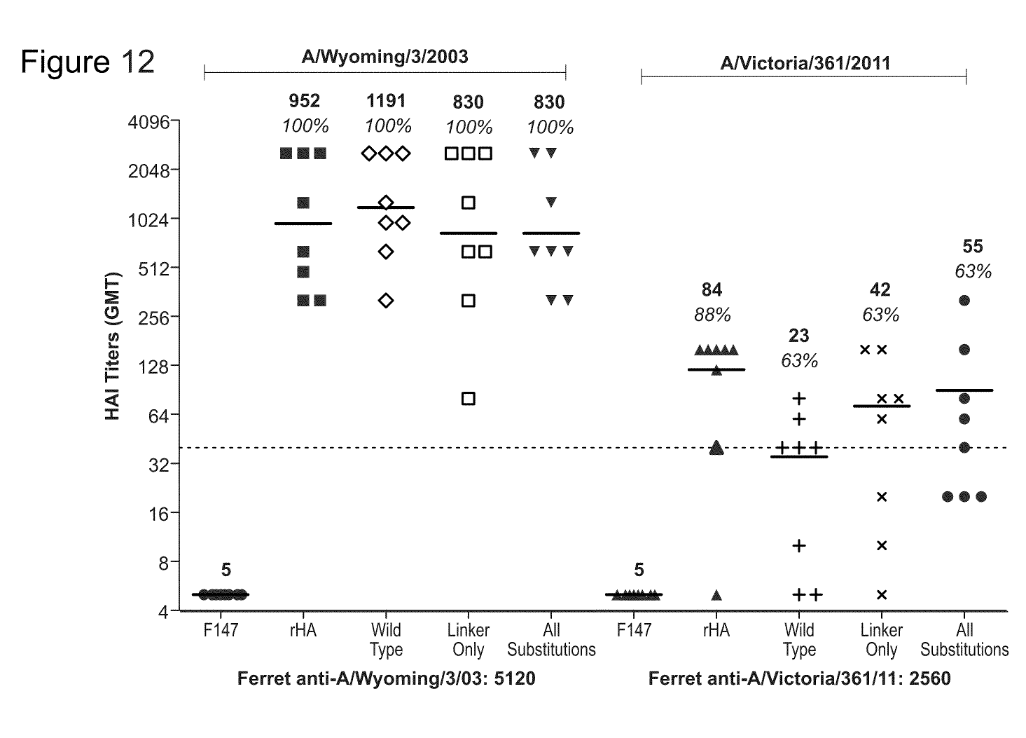

Groups of 8 BALB/c mice were immunized s.c. with indicated candidates or baculovirus-produced HA0 (Protein Sciences) with a 5 μg dose on days 0 and 21, and then bled on day 35. Serum samples were subjected to the HAI test using matched virus: A/Wyoming/3/2003 or A/Victoria/316/2011. Controls included HA0 WY (Protein Sciences), delivered in Titermax, at 6 μg, and F 147 formulation buffer. Data represent results of individual mice with geometric mean titers (GMTs) shown as bars and red text above each group (FIG. 12). Seroconversion rates are shown in green. Candidates are listed as Wild Type, Linker Only (=2 negatively charged substitutions in the linker), and All Substitutions (=3 globular head substitutions and 2 linker substitutions).

The Wyoming strain, is strongly immunogenic, with seroconversion rates of 100% and very similar geometric mean titers (GMT) for all test articles at a 5 μg dose (FIG. 12). The Victoria strain is less immunogenic. In this case the seroconversion rates of the flagellin-fusion vaccines is the same (63%) for all groups, but the GMT modestly increases from 23 for the wild type, to 42 with the addition of the substitutions in the linker (42), to 55 with the addition of the with globular head and linker substitutions.

While this invention has been particularly shown and described with references to example embodiments thereof, it will be understood by those skilled in the art that the various changes in form and details may be made therein without departing from the scope of the invention encompassed by the appended claims.

What is claimed is:

1. An immunologic fusion protein comprising flagellin and HA linked together by a linker wherein the HA has been modified such that its core residues within the interior of the HA head domain is less hydrophobic than wild-type HA and the surface of the HA has been modified to have a lower pI.

2. The immunologic fusion protein of claim 1, wherein the HA is a H3 HA.

3. The immunologic fusion protein of claim 2, wherein the H3 HA is from an influenza virus that infects humans.

4. The immunologic fusion protein of claim 3, wherein phenylalanine is changed to tyrosine at residue 47 and isoleucine is changed to valine at residue 139 and lysine is changed to serine at residue 52.

5. The immunologic fusion protein of claim 3, further comprising introducing one or more negative charges in the linking region.

6. The immunologic fusion protein of claim 5, wherein asparagine is changed to aspartic acid at residue 272 and glutamine is changed to glutamic acid at residue 275.

7. An immunologic fusion protein comprising flagellin and HA linked together by a linker wherein the HA has been modified such that its core residues within the interior of the HA head domain is less hydrophobic than wild-type HA.

8. The immunologic fusion protein of claim 7, wherein the HA is a H3 HA.

9. The immunologic fusion protein of claim 8, wherein the H3 HA is from an influenza virus that infects humans.

10. The immunologic fusion protein of claim 9, wherein the amino acids at residues 47 and 139 are changed to include residues that are less hydrophobic than the wild type residues.

11. The immunologic fusion protein of claim 10, wherein phenylalanine is changed to tyrosine at residue 47 and isoleucine is changed to valine at residue 139.

12. An immunologic fusion protein comprising flagellin and HA linked together by a linker wherein the HA has been modified such that the pI of the HA is reduced.

13. The immunologic fusion protein of claim 12, wherein the HA is a H3 HA.

14. The immunologic fusion protein of claim 13, wherein the H3 HA is from an influenza virus that infects humans.

15. The immunologic fusion protein of claim 14, wherein the amino acid at residue 52 is altered from the wild type amino acid.

16. The immunologic fusion protein of claim 15, wherein the amino acid at residue 52 is changed from lysine to serine.

17. An immunologic fusion protein comprising flagellin and HA linked together by a linker wherein the linker comprises two negatively charged amino acids.

18. The immunologic fusion protein of claim 17, wherein the negatively charge amino acids are at residues 272 and 275 of HA1-1L.

19. The immunologic fusion protein of claim 17, wherein the amino acids at residues 272 and 275 are aspartic acid glutamic acid, respectively.

20. The immunologic fusion protein of claim 17, wherein the HA comprises a tyrosine at residue 47, a valine at residue 139 and a serine at residue 52.
